(19) United States
(12) Patent Application Publication
(10) Pub. No.: US 2003/0068274 A1
(43) Pub. Date: Apr. 10, 2003

(54) Method and device for detection of substances in vital tissue

(76) Inventors: Holger Jungmann, Gelsenkirchen (DE); Martin Schmidt, Oberdorf (DE); Michael Schietzel, Herdecke (DE)

Correspondence Address: KATTEN MUCHIN ZAVIS ROSENMAN, 575 MADISON AVENUE, NEW YORK, NY, 10022-2585, US

(21) Appl. No.: 10/176,520

(22) Filed: Jun. 20, 2002

(30) Foreign Application Priority Data
Jun. 20, 2001 (DE) 101 29 754.8

(51) Int. Cl. A61K 49/00; A61B 8/00

(52) U.S. Cl. 424/9.6; 600/317

(57) ABSTRACT

The invention relates to a method and device for the detection, in particular for determining the concentration, of substances in vital tissue. The object of the invention is to create a method and device for the detection of substances in vital tissue, this permitting the reliable qualitative and quantitative detection of such substances in a physiologically safe manner. The object of the invention is achieved by a method for the detection of substances in vital tissue in which light of a predetermined wavelength is directed onto the tissue in such a manner that the light penetrates into the tissue; at least some of the light escaping from the tissue is captured and the reflected light is analyzed with an association being made between its wavelength and intensity; and the thus determined properties of the reflected light are compared with at least one reference system, the presence and/or concentration of a substance being deduced on the basis of a correlation with the reference system.

The invention relates to a method and device for the detection, in particular for determining the concentration, of substances in vital tissue.

The object of the invention is to create a method and device for the detection of substances in vital tissue, this permitting the reliable qualitative and quantitative detection of such substances in a physiologically safe manner.

The object of the invention is achieved by a method for the detection of substances in vital tissue in which light of a predetermined wavelength is directed onto the tissue in such a manner that the light penetrates into the tissue; at least some of the light escaping from the tissue is captured and the intensity of the reflected light, or the optical density of the illuminated medium, is determined with an association being made with its wavelength; and the thus determined intensity distribution of the reflected light or optical density is compared with at least one reference system, the presence and/or concentration of a substance being deduced on the basis of the result of the comparison.

This makes it possible in advantageous manner to detect a variety of substances in vivo, non-invasively and to match any medical relief or treatment measures precisely to the physiological condition of the patient. In particularly advantageous manner, it is also possible to detect substances in the circulatory system of the patient because of disease and to adjust medication on the basis thereof. Also in advantageous manner, it is possible to detect psychoactive substances, their decomposition products, illegal substances as well as drugs and also their metabolites.

According to a particularly preferred embodiment of the method, the references, for example in the form of reference spectra specified in the manner of data records, are adjusted in such a manner that they—or the correlations therewith—for example in the form of agreements/deviations therefrom—are each indicative of a certain substance or group of substances.

Alternatively thereto—or in particularly advantageous manner also in combination with the previously specified measure—it is also possible to provide a plurality of reference systems, particularly a multiplicity of, for example, stored reference spectra available in the manner of data records, the presence and/or concentration of a substance being deduced depending on the fulfillment of predetermined relationships with the reference systems, particularly reference spectra and the measured spectral distribution.

In advantageous manner, a substance is identified and the concentration of the detected substance is deduced on the basis of the intensity of selected wavelength regions. According to a particularly preferred embodiment of the method according to the invention, said wavelength regions are selected with a view to an expected classification result.

The spectrum of the light radiated onto the tissue extends preferably over a wavelength range from 200 to 800 nm. For the detection of psychoactive substances, it is particularly suitable to employ a wavelength window between 220 and 400 nm.

It is possible to fix the spectral width of the light radiated onto the tissue at a value smaller than the bandwidth of the analysis range, for example 60 nm. In particular, it is possible to use substantially monochromatic, in particular coherent light and to modulate its frequency successively over the analysis range, for example from 220 to 400 nm. Modulation is performed preferably in such a manner that the wavelength of the light is changed successively in steps of 0.3 nm. If using monochromatic light, it is possible for fluorescence or band shift effects, caused by the substance to be detected, to be used in the detection thereof. Alternatively to the use of monochromatic light, it is also possible to employ white light in this procedure.

According to a particularly preferred embodiment of the invention, the comparison of the measured wavelengths/intensity distribution is performed on the basis of a correlation consideration, the presence and/or concentration of a substance or groups of substances being able to be deduced depending on the fulfillment of a correlation relationship.

Preferably, there are provided a plurality of reference bases used for the identification of amphetamines, benzodiazepines, cannabinoids, methadone, antiepileptics and ecgonines.

Furthermore, the reference bases also preferably include data records for the identification of heroin derivatives, cocaine, LSD, nor-LSD, opiates, buprenorphine, gabapentine, carbamazepine, oxcarbazepine, antidepressants, neuroleptics, barbiturates and antibiotics.

According to a particularly preferred embodiment of the invention, the reflected light is measured at places on the body with different degrees of blood oxygenation. This makes it possible to determine the oxygen content of the blood and the haemoglobin content of the blood and to take account of influences of the degree of blood oxygenation in the evaluation of the signal.

The reference system preferably makes available reference data provided for the identification of amphetamines. In particularly advantageous manner, the reference system makes available a plurality of reference data provided for the identification of benzodiazepines. Also in advantageous manner, the reference system makes available a plurality of reference bases for the identification of methadone enantiomers and racemic mixture thereof. Also in advantageous manner, the reference system makes available a plurality of reference bases for the identification of heroin. Also in advantageous manner, the reference system makes available a plurality of reference bases suitable for the identification of buprenorphine, gabapentine, carbamazepine, zolpideme, zopiclone, dextromethorphane, doxepine, promethazine and/or oxcarbazepine. The reference system also preferably makes available a plurality of reference bases for the identification of cocaine and ecgonines. In advantageous manner, the reference system can also make available a plurality of reference bases for the identification of LSD and nor-LSD. The reference system also preferably makes available a plurality of reference bases for the identification of opiates. The reference system also preferably makes available a plurality of reference bases for the identification of cannabinoids. Preferably, the reference system makes available in particular a plurality of reference bases for the identification of metabolites of the aforementioned substances. The reflected light is captured preferably at at least two differently located measuring points or measuring points different with regard to capillarization features, or blood oxygenation. Preferably, the method is performed in such a manner that the dependence of the reflectance spectrum on the degree of oxygenation is included in signal evaluation.

With regard to the device, the initially indicated object of the invention is also achieved by a device for the detection of substances in vital tissue, with a light source for producing light of a predetermined spectrum and for radiating the light onto the vital tissue in such a manner that the light penetrates into the tissue; with a light-capturing means for capturing at least some of the light reflected from the tissue; with a measuring means for measuring the intensity of the reflected light with an association being made with the wavelength; and with a comparison means for comparing the calculated intensity distribution of the reflectance spectrum with at least one reference data record, the presence and/or concentration of a substance or group of substances being deduced on the basis of the result of the comparison.

In particularly advantageous manner, the device comprises an evaluation system which is designed in such a manner that it adaptively changes the algorithm, the evaluation procedure and/or the reference system with the inclusion of the measured data. In advantageous manner, the device is of such design that there is self-calibration on the basis of the measured data. In advantageous manner, the device is of such design that a functional check is performed on the basis of the measured data. In advantageous manner, the device is of such design that there are probability statements on the presence of substances which may possibly be confused with each other. Also in advantageous manner, the device is of such design that there is an automatic error analysis with each measurement. In advantageous manner, the device is of such design that there is an automatic error analysis which, for example, minimizes the probability of future possible measuring errors or incorrect substance identification.

In particularly advantageous manner, the substances to be detected are identifiable by fluorescence effects. Light, which is radiated, preferably in a wavelength range from 200 to 400 nm, onto the examined tissue in vivo, enriches the energy content of the substances under examination. At a certain time interval, which may in certain cases also be substance-specific, the substances release some of the said energy at offset, in particular higher, wavelength ranges between 240 and 1000 nm. Enrichment energy and radiation energy have different wavelength ranges. For this reason, enrichment energy and radiation energy can be distinguished. The radiation energy is lower than the enrichment energy. This means that the radiation spectrum (=fluorescence spectrum) is in a higher wavelength range. This lower-energy radiation energy results in the so-called fluorescence spectrum and can be measured by means of a measuring means.

The thus produced spectra result in characteristic signals in a wavelength range between 240 and 1000 nm. The substances differ within the spectrum through characteristic portions. In particular, the combined observation of selected portions of the spectral range of the fluorescence spectrum makes it possible—also in the case of high superposition of the spectrum—to achieve sufficiently reliable identification of the substances to be detected. Portion-specific features and combination criteria can be specified on a substance-specific basis and can, in advantageous manner, be adaptively optimized—for example, in consideration of spectrum-specific features of the particular environment. Such a procedure makes it possible for the substances in question to be distinguished with high reliability in view, for example, of their characteristic distribution of the spectrum maxima.

The measured fluorescence data are preferably evaluated by means of digital processing by being compared with reference data. The comparison of data, particularly correlation properties, results in the identification of a substance.

The quantification of an identified substance results from the optical density of the irradiated medium. There are different optical densities depending on the concentration of a substance contained in the irradiated medium.

There are specific signals for different substances. These signals are determined in the form of fluorescence spectra. The fluorescence spectra can be associated with the substances which are to be detected.

Further details of the invention will become apparent from the following description in conjunction with the drawings, in which:

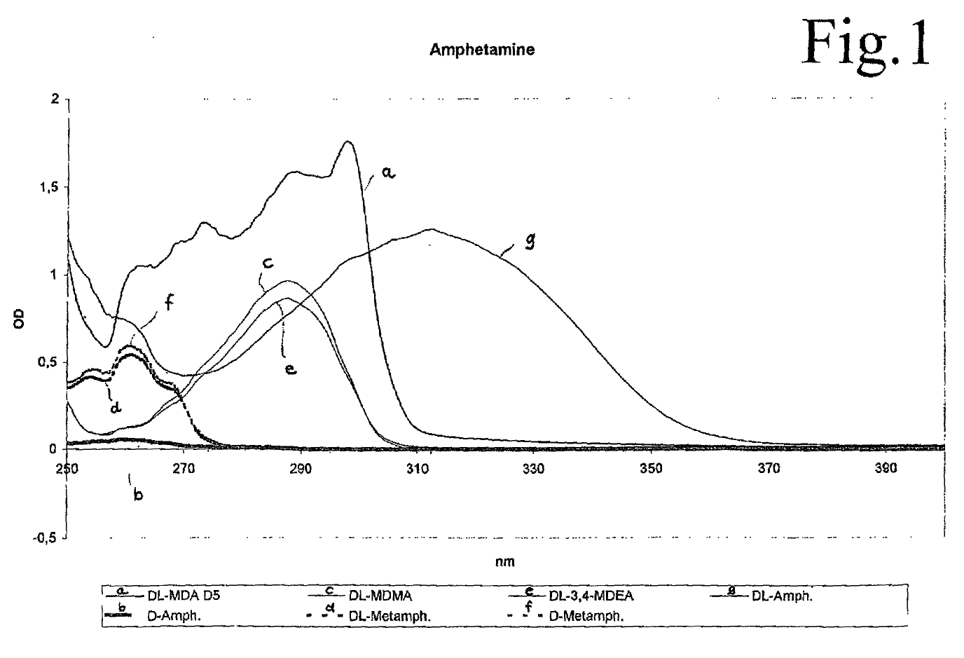

FIG. 1 shows a spectrum characteristic with regard to the intensity distribution of the light reflected from vital tissue, in a wavelength range from 240 to 390 nm, for amphetamines;

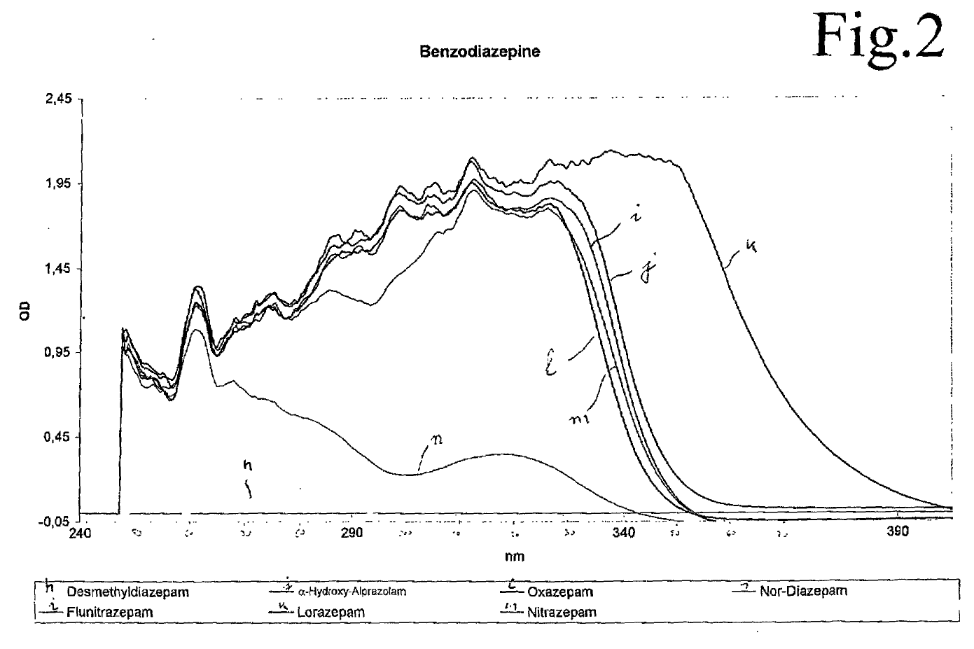

FIG. 2 shows a spectrum characteristic with regard to the intensity distribution of the light reflected from vital tissue, in a wavelength range from 240 to 410 nm, for benzodiazepines;

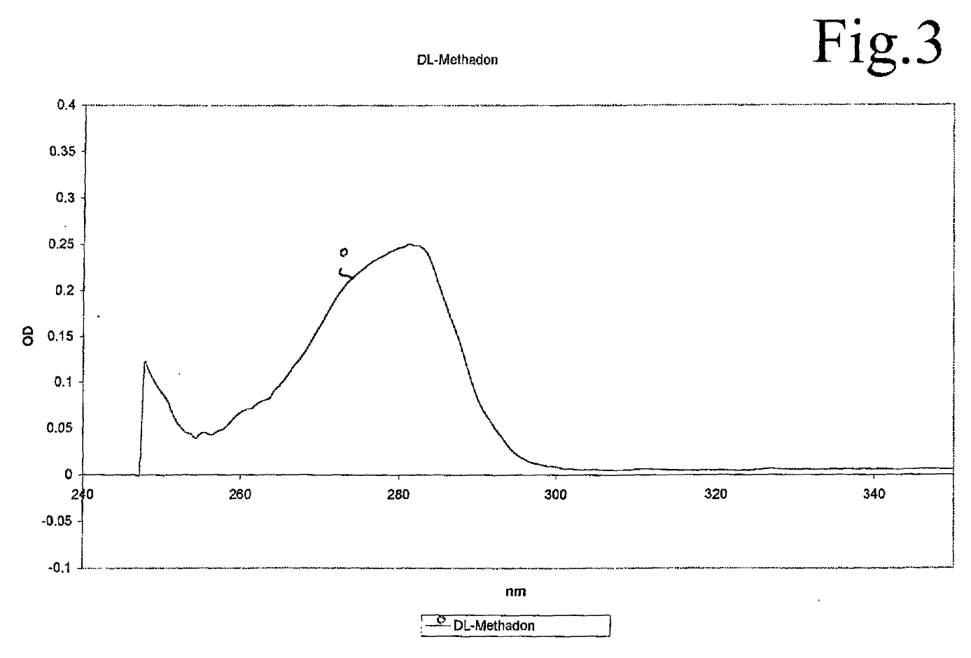

FIG. 3 shows the intensity distribution of the light reflected from tissue, in a wavelength range from 240 to 350 nm, for DL-methadone (500 ng/ml);

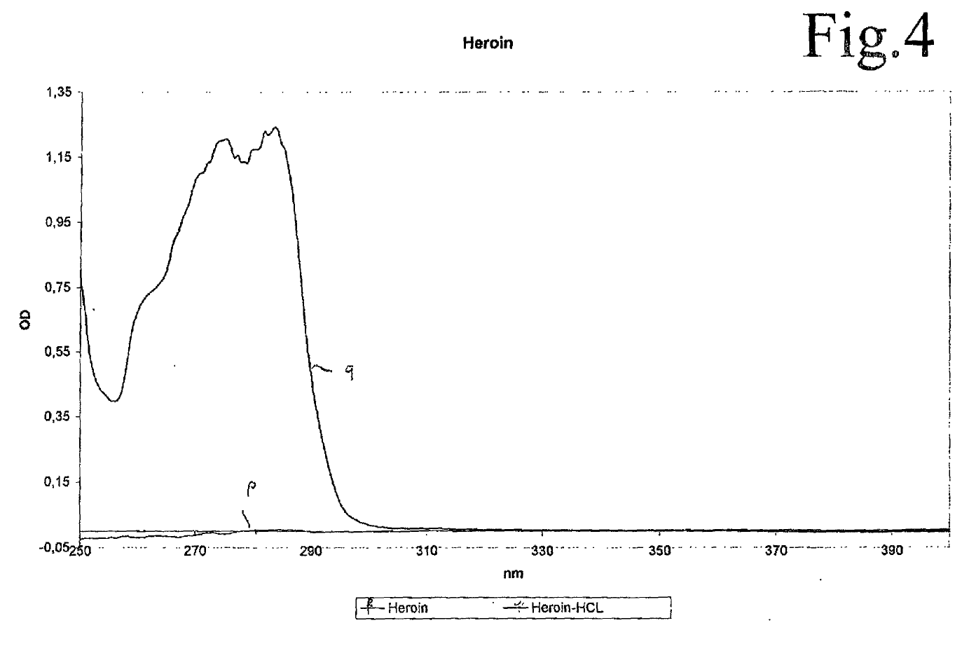

FIG. 4 shows a spectrum characteristic with regard to the intensity distribution of the light reflected from vital tissue, in a wavelength range from 250 to 390 nm, for heroin;

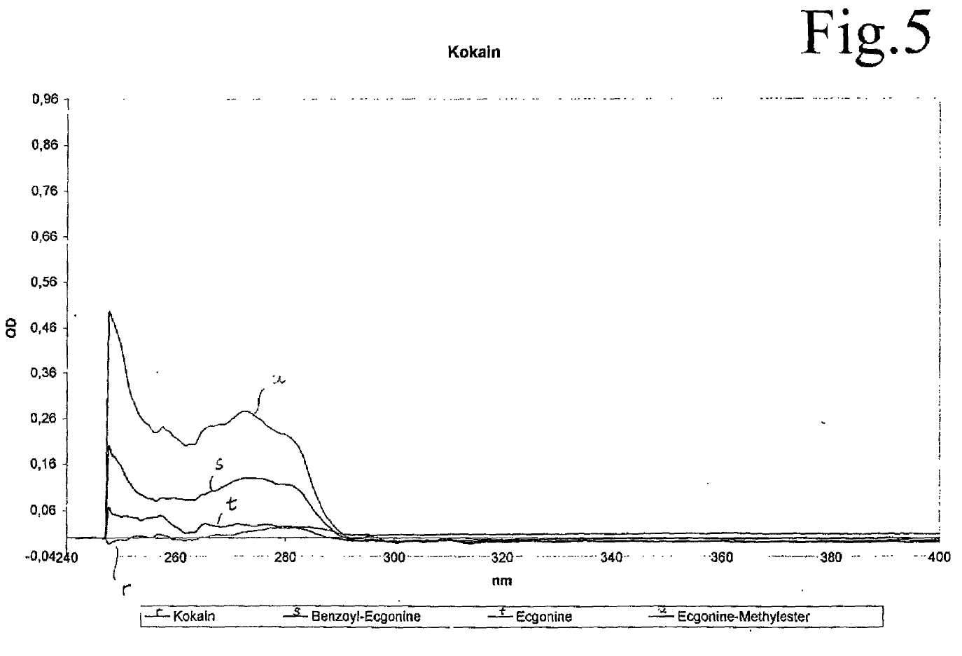

FIG. 5 shows a spectrum characteristic with regard to the intensity distribution of the light reflected from vital tissue, in a wavelength range from 240 to 400 nm, for cocaine;

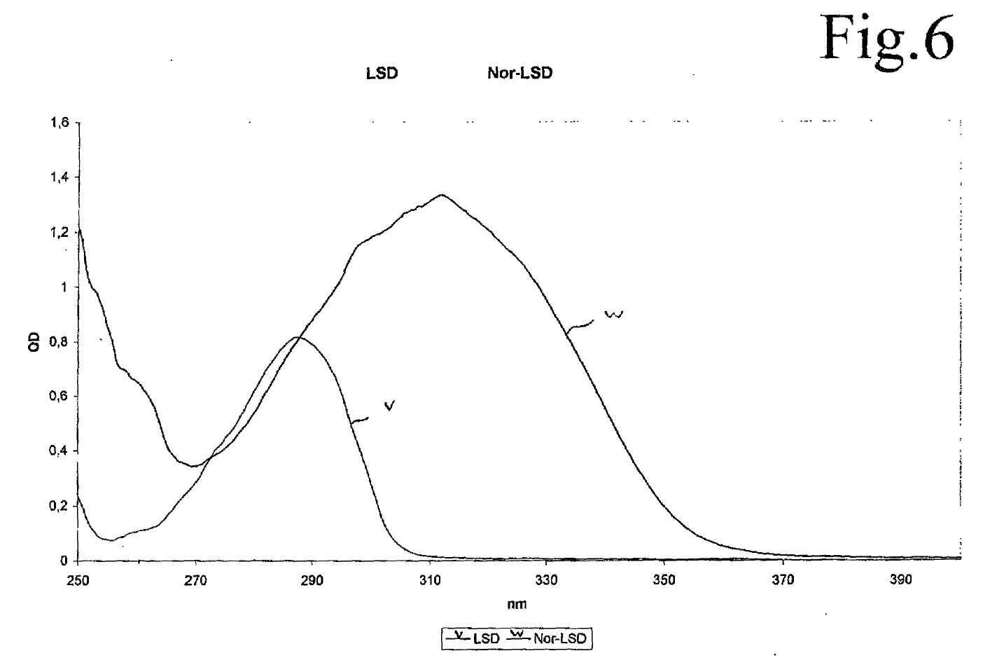

FIG. 6 shows a spectrum characteristic with regard to the intensity distribution of the light reflected from vital tissue, in a wavelength range from 250 to 390 nm, for LSD and nor-LSD;

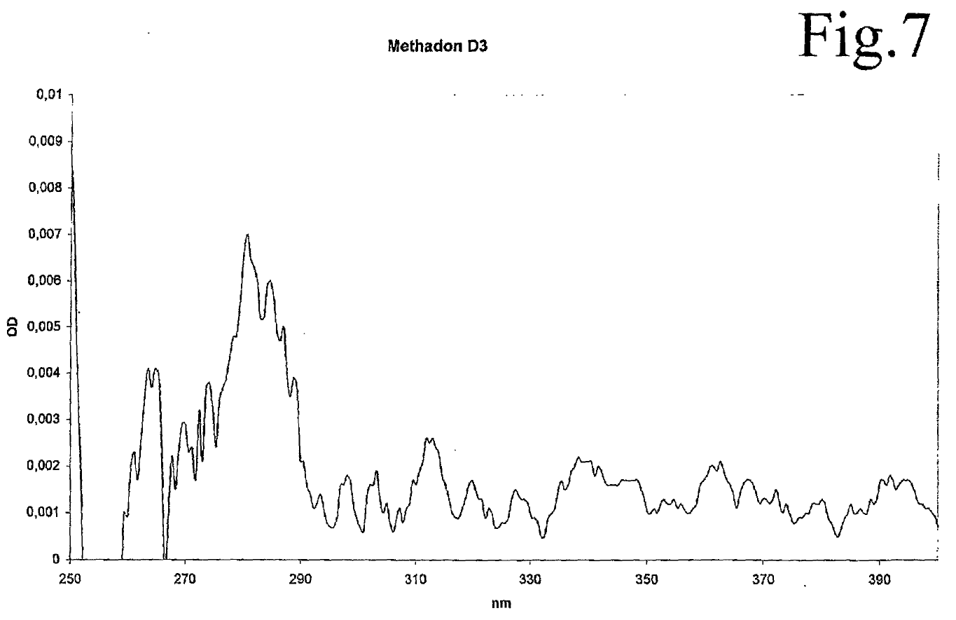

FIG. 7 shows a spectrum characteristic with regard to the intensity distribution of the light reflected from vital tissue, in a wavelength range from 250 to 390 nm, for methadone D3;

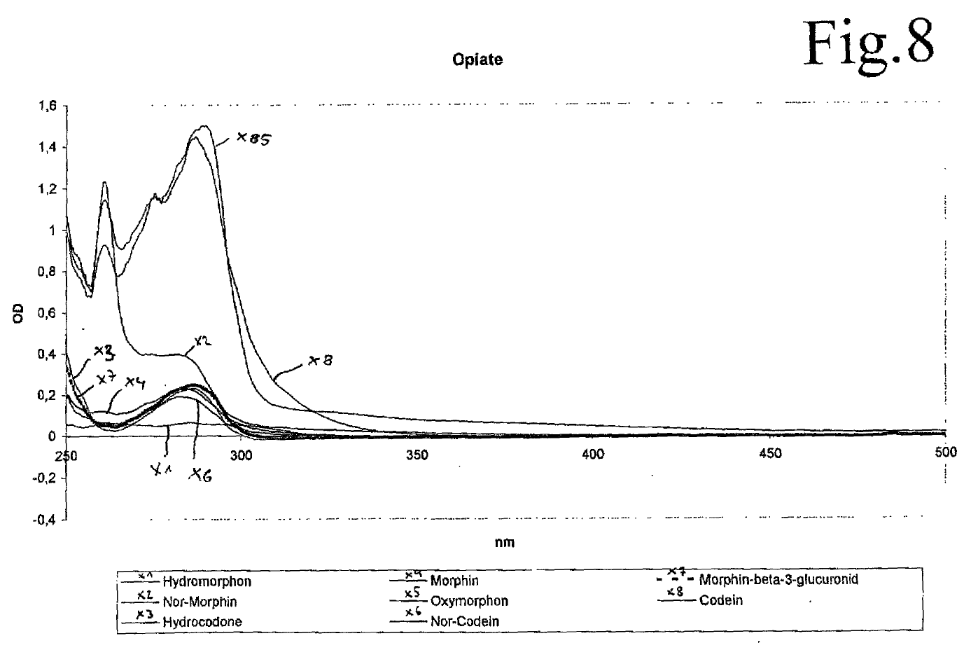

FIG. 8 shows a spectrum characteristic with regard to the intensity distribution of the light reflected from vital tissue, in a wavelength range from 250 to 500 nm, for selected opiates;

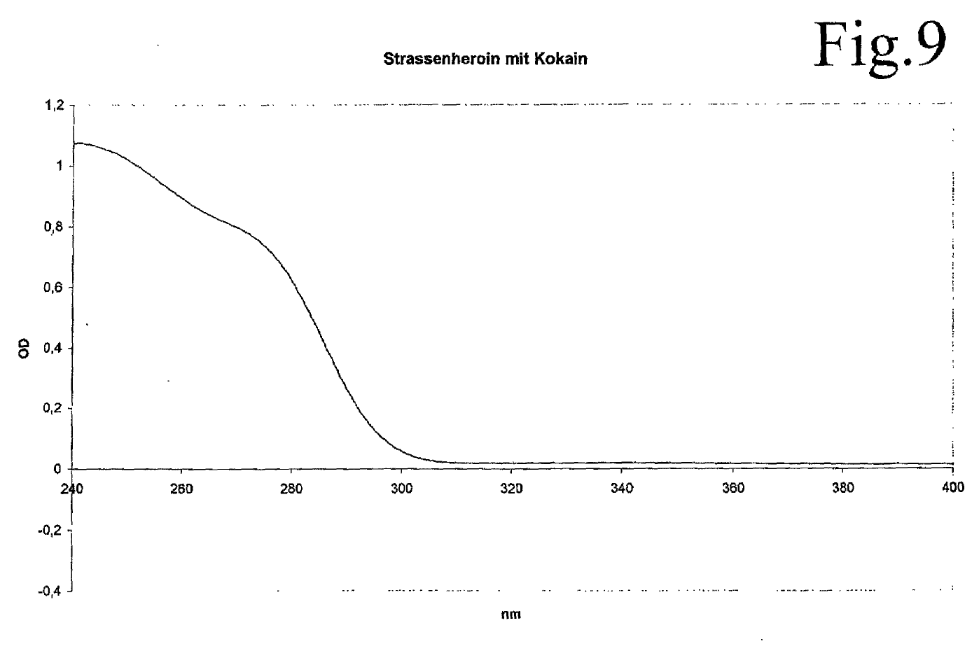

FIG. 9 shows a spectrum characteristic with regard to the intensity distribution of the light reflected from vital tissue, in a wavelength range from 240 to 400 nm, for street heroin with cocaine;

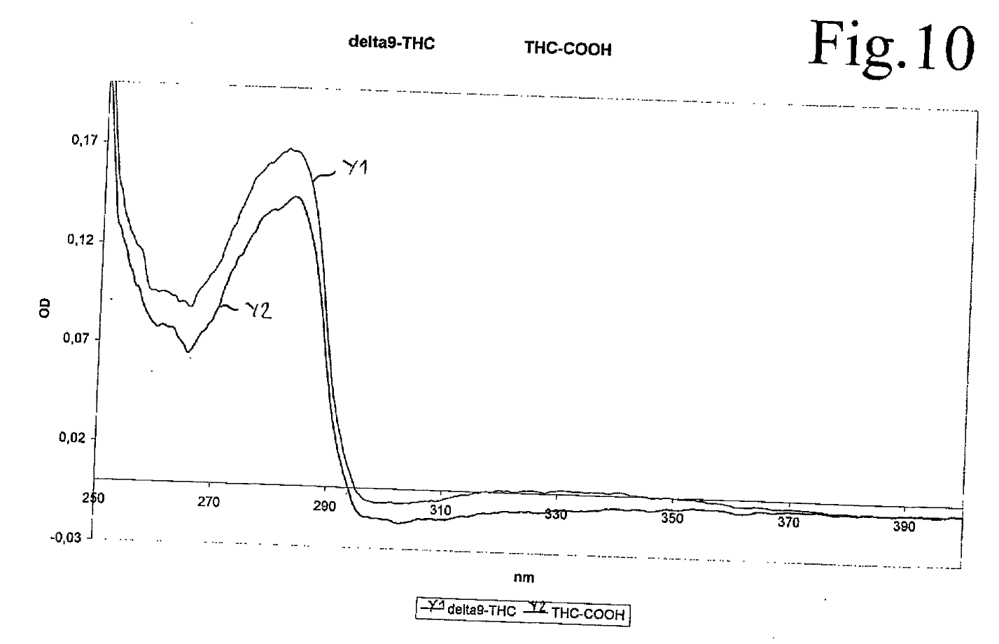

FIG. 10 shows a spectrum characteristic with regard to the intensity distribution of the light reflected from vital tissue, in a wavelength range from 250 to 390 nm, for delta9-THC;

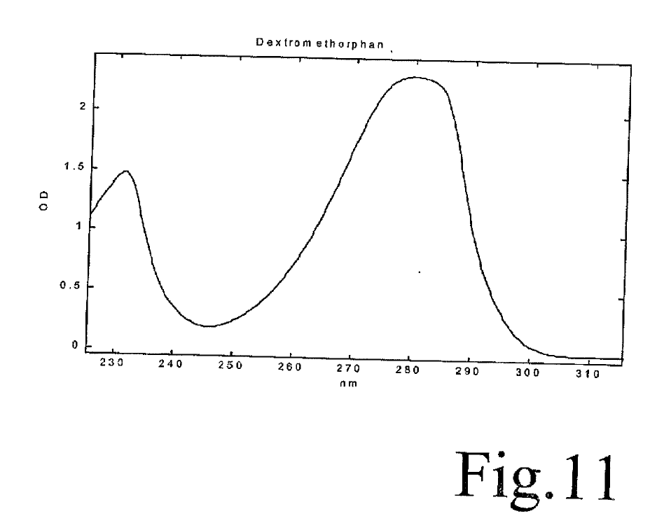

FIG. 11 shows a spectrum characteristic with regard to the intensity distribution of the light reflected from vital tissue, in a wavelength range from 220 to 310 nm, for dextromethorphane;

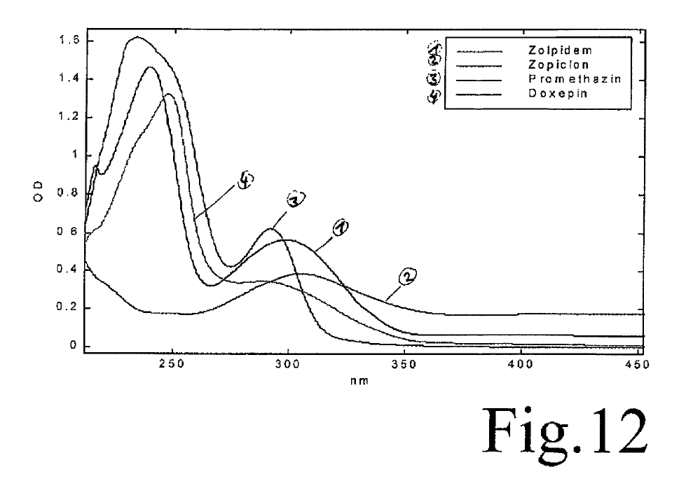

FIG. 12 shows a spectrum characteristic with regard to the intensity distribution of the light reflected from vital tissue, in a wavelength range from 220 to 450 nm, for the benzodiazepine analogs zolpideme and zopiclone, the neuroleptic promethazine and the tricyclic antidepressant doxepine;

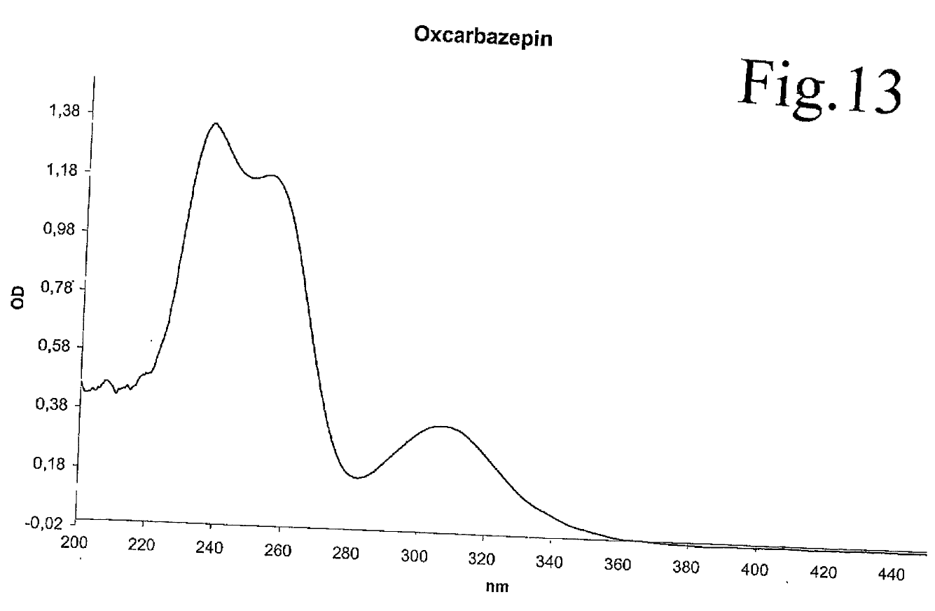

FIG. 13 shows a spectrum characteristic with regard to the intensity distribution of the light reflected from vital tissue, in a wavelength range from 200 to 440 nm, for oxcarbazepine;

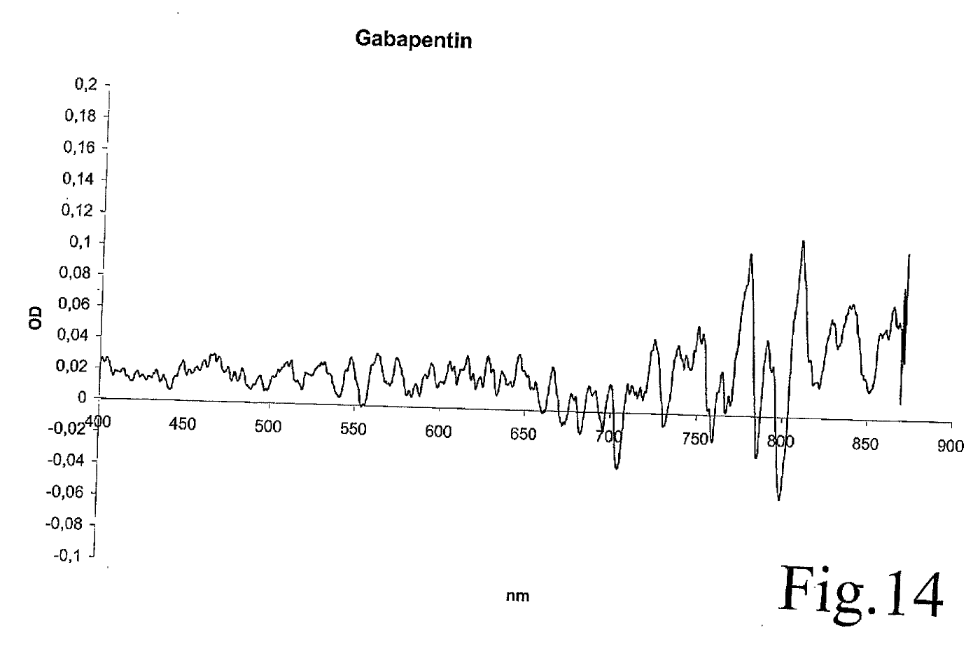

FIG. 14 shows a spectrum characteristic with regard to the intensity distribution of the light reflected from vital tissue, in a wavelength range from 400 to 870 nm, for gabapentine;

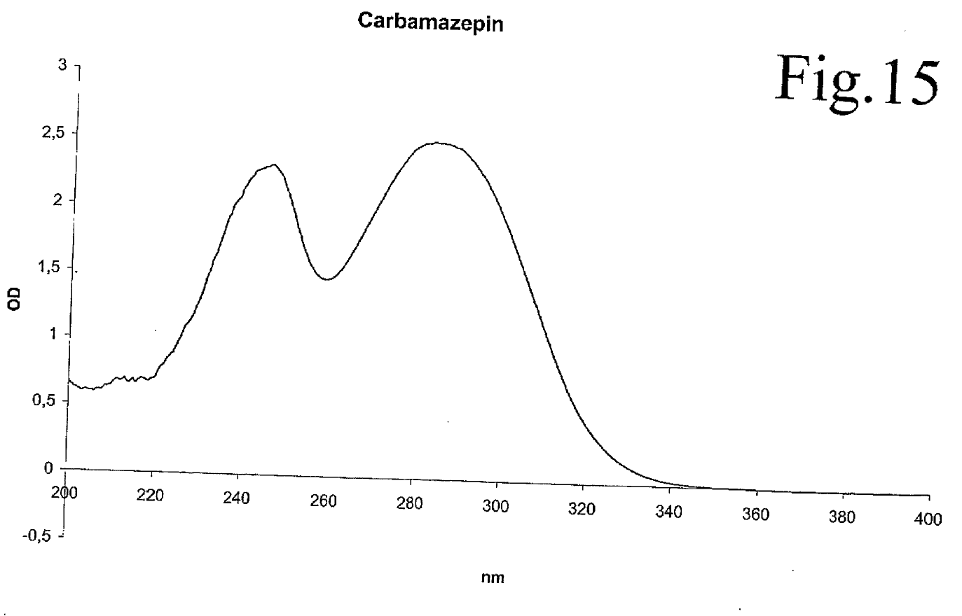

FIG. 15 shows a spectrum characteristic with regard to the intensity distribution of the light reflected from vital tissue, in a wavelength range from 200 to 400 nm, for carbamazepine;

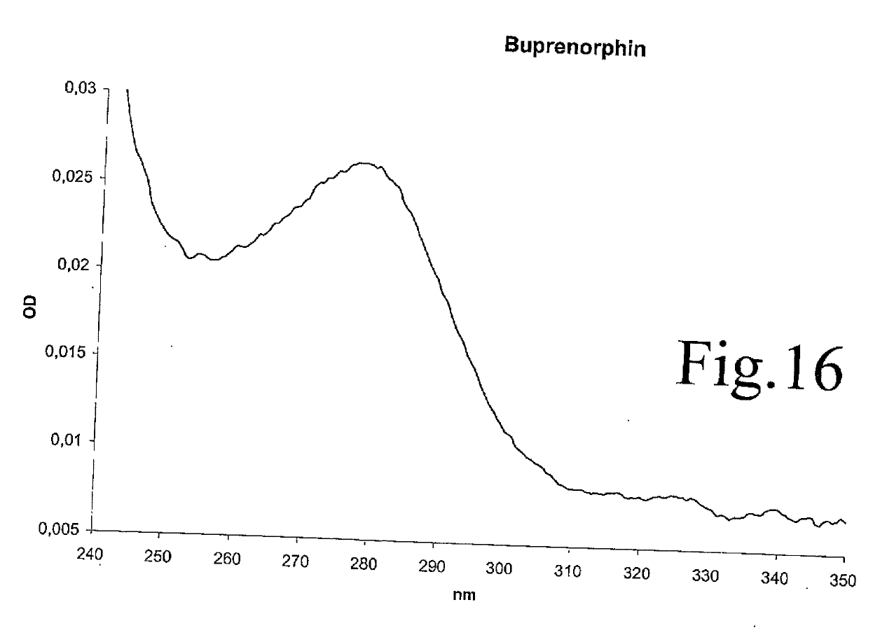

FIG. 16 shows a spectrum characteristic with regard to the intensity distribution of the light reflected from vital tissue, in a wavelength range from 200 to 350 nm, for buprenorphine;

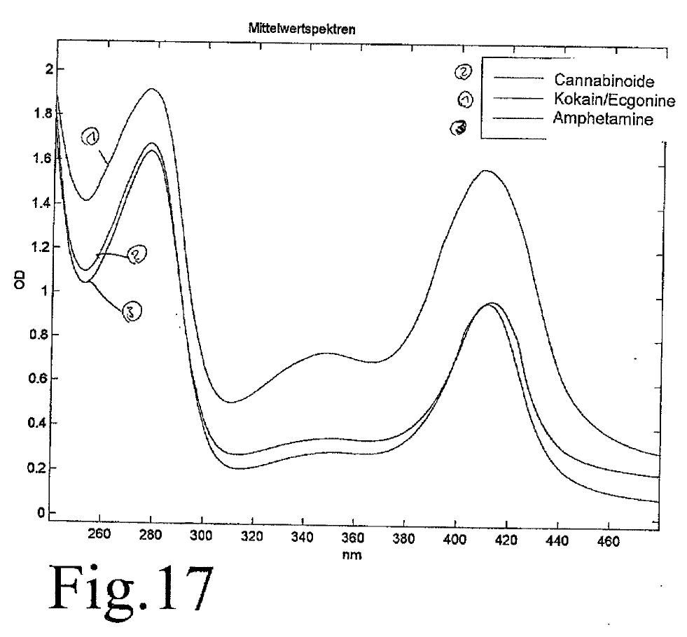

FIG. 17 shows mean value spectra of human blood for a wavelength range from 240 to 480 nm for cannabinoids, cocaine with its decomposition products (ecgonines) and amphetamines;

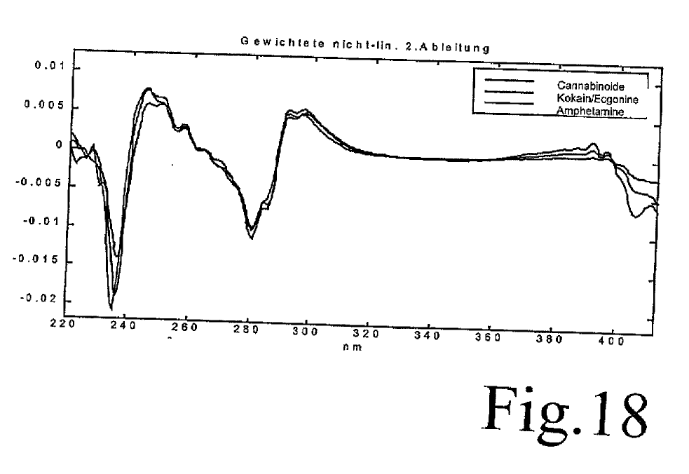

FIG. 18 shows the weighted, non-linear second derivatives of cannabinoids, cocaine with its decomposition products (ecgonines) and amphetamines in a wavelength range from 220 to 420 nm;

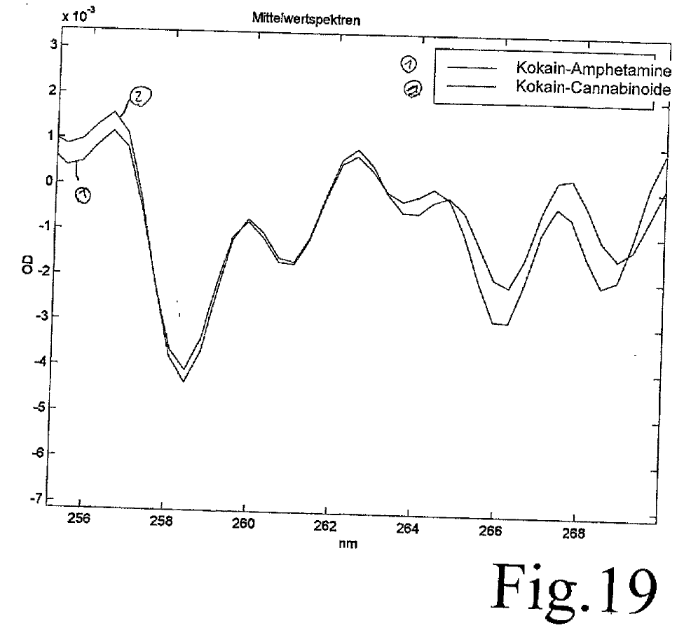

FIGS. 19-21 shows the pairwise gradients of the weighted, non-linear derivatives e.g. for the group of cocaine and ecgonines, the group of amphetamines and the group of cannabinoids in a wavelength range from 220 to 420 nm;

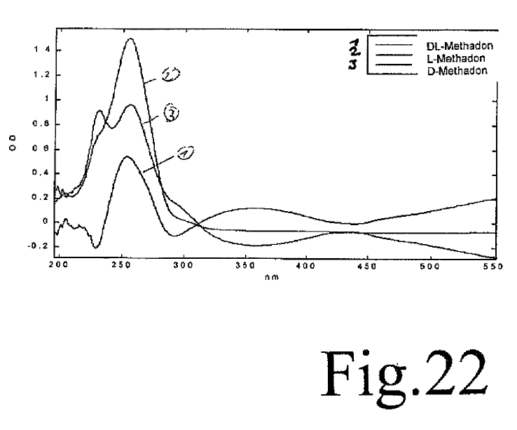

FIG. 22 shows a spectrum characteristic with regard to the intensity distribution of the light reflected from vital tissue, in a wavelength range from 200 to 550 nm, for DL-methadone, L-methadone and D-methadone;

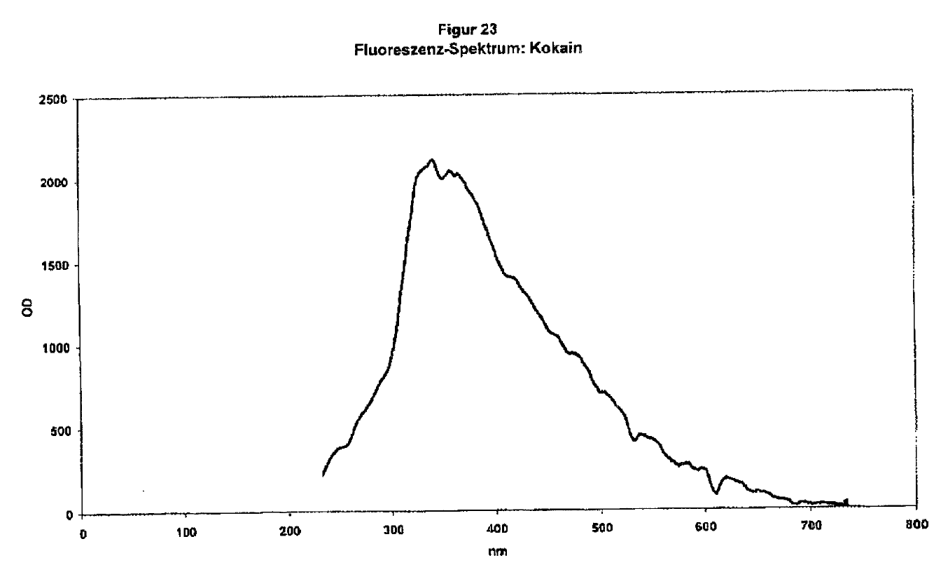

FIG. 23 shows for cocaine the fluorescence spectrum of light reflected from vital tissue, in a wavelength range from 240 to 740 nm;

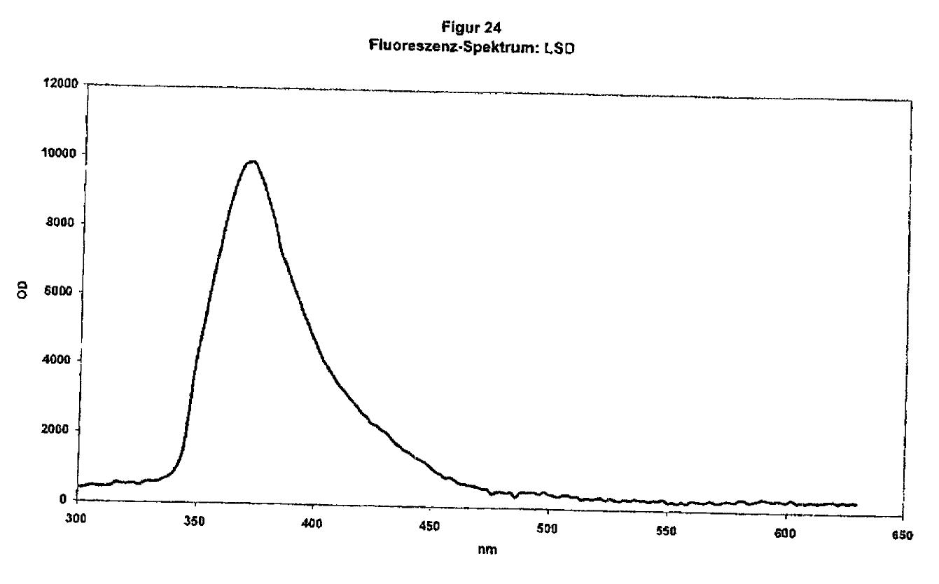

FIG. 24 shows for LSD the fluorescence spectrum of light reflected from vital tissue, in a wavelength range from 300 to 630 nm;

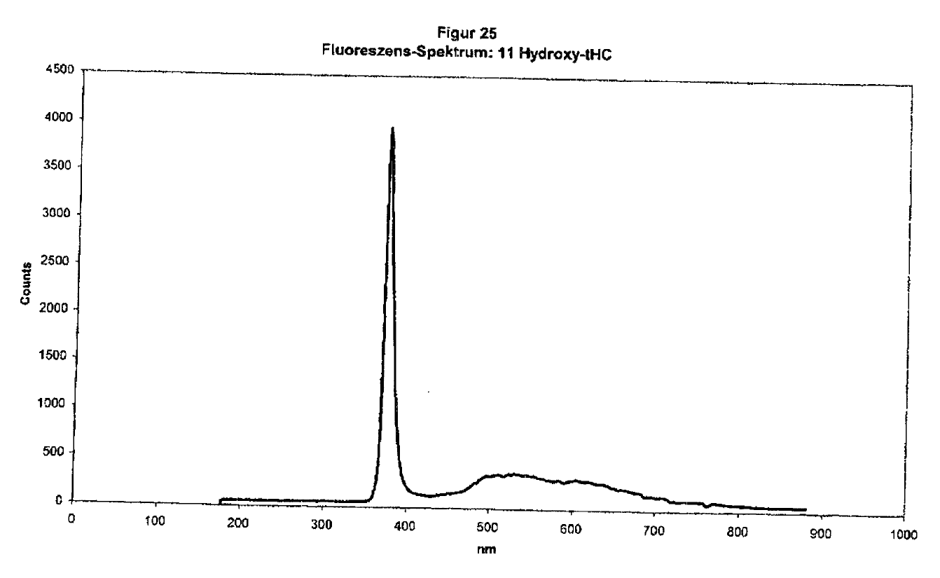

FIG. 25 shows for 11 hydroxy-THC the fluorescence spectrum of light reflected from vital tissue, in a wavelength range from 240 to 880 nm;

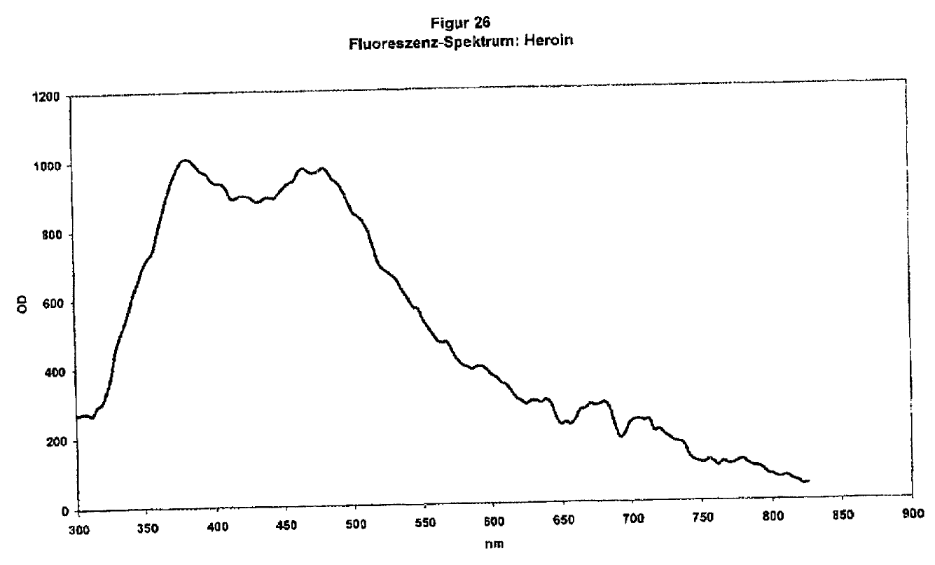

FIG. 26 shows the fluorescence spectrum of heroin in a light reflected from vital tissue, in a wavelength range from 300 to 825 nm;

FIG. 1 shows a spectrum characteristic with regard to the intensity distribution of the light reflected from vital tissue and measured by a measuring means, for different amphetamines.

Graph a shows the reflected light spectrum for DL-MDA D5. Graph a exhibits at a wavelength of 256 nm a characteristic local minimum. At wavelengths of 275 nm, 250 nm and 296 nm there are clearly defined local maxima of the optical density of the measured reflected light. The optical density of the measured reflected light is 0.6 for the wavelength of 256 nm, 1.25 for the wavelength 274 nm, 1.5 for the wavelength of 286 nm and 1.7 for the wavelength of 298 nm.

Graph b describes the reflected light spectrum for D-amphetamine in a concentration of 500 ng/ml. Graph b, characteristic of D-amphetamine, exhibits a local maximum at a wavelength of 260 nm. In comparison with the other described amphetamines, even in the herein shown concentration of 500 ng/ml, D-amphetamine is reliably detectable in the wavelength window from 250 to 400 nm inasmuch as the spectrum characteristic of D-amphetamine differs significantly from the spectra otherwise typical of amphetamines and exhibits a maximum in particular at 260 nm.

Graph c shows a characteristic reflected light spectrum for DL-MDMA in a concentration of 500 ng/ml. Graph c has a characteristic absolute maximum at a wavelength of 286 nm. Graph c exhibits no further local extreme values in the range from 260 to 310 nm. Graph c is characterized by a bell-shaped curve which is essentially symmetrical with respect to the absolute maximum at 286 nm. The absolute maximum of DL-MDMA is situated in the range of a concentration which is of relevance for the central nervous system, approximately in the range of the local maximum of DL-MDA (Graph a).

Graph d shows a characteristic curve of the optical density over the wavelength for DL-metamphetamine. At a wavelength of 257 nm there is a characteristic local minimum and at a wavelength of 261 nm there is a characteristic absolute maximum. In the wavelength range of 265 nm as well as at 269 nm there are maxima characteristic of the second derivative of the optical density with respect to the wavelength.

Graph e illustrates a characteristic reflected light spectrum for DL-3,4-MDEA at a concentration of 500 ng/ml. Graph e is characterized by a local minimum at a wavelength in the range of 255 nm. At a wavelength in the range of 286 nm there is a local maximum similarly to DL-MDMA (500 ng/ml). Beyond a wavelength of 310 nm the optical density of the reflected light for DL-3,4-MDEA (500 ng/ml) is almost 0.

Graph f describes the reflected light spectrum for D-metamphetamine (500 ng/ml). At a wavelength of 254 nm there is a local maximum. At a wavelength of 257 nm there is a local minimum. At a wavelength of 260 nm there is an absolute maximum. At wavelengths of 265 nm and 269 nm there are maxima with regard to the derivative of the optical density with respect to the wavelength.

Graph g shows a reflected light spectrum significant for a DL-amphetamine at a concentration of 500 ng/ml. Of significance are a local minimum at a wavelength of 270 nm as well as a minimum at a wavelength of 262 nm and a maximum at a wavelength of 312 nm.

FIG. 2 shows the reflected light spectrum for a plurality of psychoactive substances belonging to the group of benzodiazepines. Graph h describes the reflected light spectrum for desmethyldiazepam in a concentration of 500 ng/ml. In particular, the spectrum in the range from 240 to 300 nm is characteristic of this substance. Thus, there is a local minimum at a wavelength of 254 nm and an absolute maximum at a wavelength of 282 nm. Beyond a wavelength of 294 nm this substance exhibits no spectral component.

Graph i describes the characteristic reflected light spectrum for flunitrazepam (500 ng/ml).

Graph j shows the reflected light spectrum for a-hydroxy-alprazolam.

Graph k shows the reflected light spectrum for lorazepam in a concentration of 500 ng/ml.

Graph l shows the reflected light spectrum for oxazepam in a concentration of (500 ng/ml).

Graph m shows the reflected light spectrum for nitrazepam (500 ng/ml).

Graph n shows the reflected light spectrum in a wavelength range from 240 to 400 nm for nor-diazepam likewise in a concentration of 500 ng/ml.

Express reference is made to FIG. 2 with regard to the features which are characteristic of the individual reflected light spectra. The detection of the psychoactive substances in the vital tissue is accomplished preferably through the combined use of two or more correlation criteria.

FIG. 3 shows the reflected light spectrum for DL-methadone in a concentration of (500 ng/ml). There is a characteristic local minimum at a wavelength of 254 nm. At a wavelength in the range of 282 nm there is an absolute maximum with an optical density of 0.26. In the area surrounding this absolute maximum there is a bell-shaped curve of the graph.

FIG. 4 shows the reflected light spectrum for heroin in a concentration of 500 ng/ml (Graph p) as well as heroin-HCL in a concentration of 50 ng/ml (Graph q).

For heroin-HCL there is a characteristic local minimum at 256 nm. There are characteristic local maxima at 275 nm as well as at 285 nm. Between these two local maxima there is at a high level a local minimum with a wavelength of 279 nm. Further features of the reflected light spectrum of significance for heroin-HCL are directly apparent from FIG. 4 (see in particular the steeply falling edge in the range from 290 to 305 nm).

FIG. 5 shows a reflected light spectrum for the identification of cocaine and its characteristic psychoactive metabolites. Graph r shows the curve for cocaine in a concentration of 100 ng/ml. The spectrum is characterized by a low maximum at 284 nm. Graph s shows the curve of benzoyl-ecgonine 100 ng/ml; Graph t shows the reflected light spectrum for ecgonine 100 ng/ml. Graph u shows the reflected light spectrum for ecgonine-methylester in a concentration of 100 ng/ml.

FIG. 6 shows a spectral window suitable for the detection of LSD in a wavelength range from 250 to 400 nm. For LSD there is a local minimum at a wavelength of 256 nm and an absolute maximum at a wavelength of 288 nm. In the region surrounding this absolute maximum there is an essentially bell-shaped fall of the curve.

For nor-LSD (Graph w) there is a local minimum at 269 nm and an absolute maximum at 310 nm. Further curve-specific characteristics suitable for the detection of the substances LSD and nor-LSD are apparent from FIG. 6. The difference of the curve maxima between LSD and its decomposition product nor-LSD is therefore 31 nm, with the consequence that it is possible to detect the parent substance and the decomposition product.

FIG. 7 shows in high resolution a reflected light spectrum of methadone D3. The optical density reaches a maximum of 0.007 at 279 nm. The light intensity is therefore very low; the identification of this substance in the vital tissue is possible with sufficient reliability particularly with regard to the characteristics in the wavelength range from 286 to 320 nm. This reflected light spectrum exhibits characteristic features particularly at the wavelengths of 279 nm, 312 nm, 340 nm and 362 nm, this further permitting the identification of methadone D3.

FIG. 8 shows a plurality of spectra significant for the identification of opiates according to the invention. Graph x1 relates to hydromorphone in a concentration of (500 ng/ml). Graph x2 relates to nor-morphine likewise in a concentration of 500 ng/ml. Graph x3 relates to hydrocodone in a concentration of 500 ng/ml. Graph x4 shows morphine in a concentration of 50 ng/ml. Graph x5 shows oxymorphone in a concentration of 500 ng/ml. Graph x6 shows nor-codeine in a concentration of 500 ng/ml. Graph x7 shows morphine-beta-3-glucoronide in a concentration of 50 ng/ml. Graph x8 shows codeine in a concentration of 500 ng/ml.

FIG. 9 shows the reflected light spectrum for street heroin with cocaine. For this psychoactive substance there is a sufficiently indicative region in a wavelength range from 240 to 310 nm. At 270 nm there is the maximum of a superposed reflected light spectrum of cocaine. The superposed spectrum contains, in particular, additives.

FIG. 10 shows the reflected light spectrum for delta 9-THC (Graph y1) and THC-COOH (Graph y2) in a concentration of 50 ng/ml. The maxima of the spectra for the two substances are at 283 nm (y2) and 280 nm (y1). Characteristic for the differentiation of the substances is the shift of the spectra with regard to the rising phase in the range from 270 to 280 nm and the falling phase in the range from 280 to 295 nm.

FIG. 11 shows the reflected light spectrum for the psychoactive substance dextromethorphane in a concentration of 100 ng/ml. Characteristic of this substance is, in particular, the wide local minimum with an optical density at a wavelength of 246 nm and the absolute maximum of the optical density at a wavelength of 280 nm as well as the local maximum of the optical density at a wavelength of 232 nm.

FIG. 12 shows the characteristic reflected light spectra for the detection according to the invention of the psychoactive substances zolpideme, zopiclone, promethazine and doxepine. Also for these substances there is a particularly high reliability of detection especially in the wavelength range from 200 to 360 nm. The substances are clearly distinguishable. The optical density in the defined wavelength range is sufficient to permit a reliable quantitative determination of the substances. The benzodiazepine analogs zolpideme and zopiclone exhibit similar spectroscopic properties to benzodiazepine owing to their similar chemical properties.

FIG. 13 shows the spectrum of oxcarbazepine. Characteristic are a maximum at 238 nm, a maximum at 255 nm, a minimum at 281 nm and a further maximum at 308 nm.

FIG. 14 shows the spectrum for gabapentine in the wavelength range from 400 to 865 nm. The great waviness of the graph in this specific region is significant for this substance. These are not artifacts such as noise. The maxima and minima characteristic of the substance or preferably the entire spectrum is stored in digital form preferably in high resolution and made available as a reference data record.

FIG. 15 shows the characteristic spectrum for carbamazepine with a twin-peak curve. The first maximum is at 245 nm, the second maximum being at 284 nm. There is a specific minimum at 249 nm.

FIG. 16 shows the characteristic spectrum for buprenorphine in a wavelength range from 240 to 350 nm. It is possible to make out fine-waved, finely structured spectra superposed on the spectrum. The characteristic maximum at 277 nm and at 224 nm results from the photoactivity of the functional group in the molecule of the substance.

FIG. 17 shows the mean value spectra of the three different groups of substances cannabinoids, cocaine/ecgonines and amphetamines from consumers of each group of substances. At approximately 280 nm the serum proteins have maximum absorption and haemoglobin at around 420 nm. Fine structures are suppressed in this representation.

FIG. 18 shows the weighted, non-linear 2nd derivatives of the starting spectra from FIG. 17. Distinct minima can be seen at 280 nm. These are produced by serum proteins. The maxima in the curve are at 238 nm.

There is detectability and a spectrum can be unambiguously associated with a substance or group of substances particularly when pairwise gradients are formed of the weighted, non-linear derivatives e.g. for the group of cocaine and ecgonines, the group of amphetamines and the group of cannabinoids. If these gradients reveal that absorption maxima are detectable for a substance or a chemically related group of substances at certain wavelengths characteristic of that substance or group of substances, then these maxima are the result of identical substances or groups of substances.

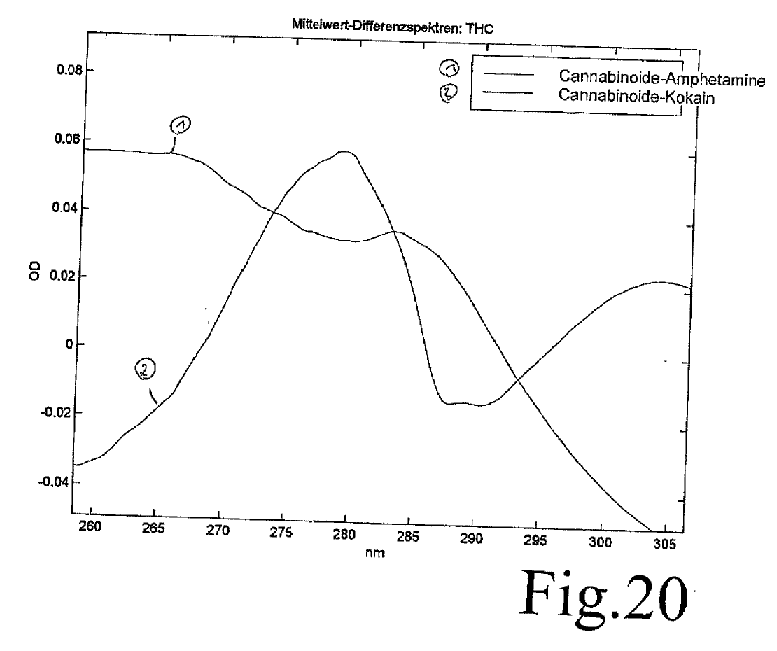

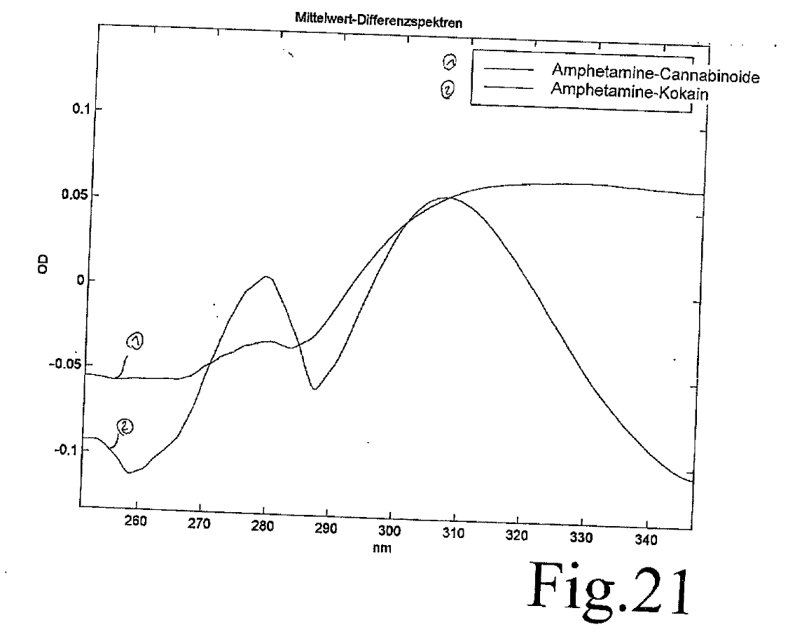

This relationship is presented for cocaine and ecgonines in FIG. 19 and for cannabinoids in FIG. 20 and for amphetamines in FIG. 21.

The formation of pairwise gradients for cocaine and ecgonines with amphetamines and the formation of pairwise gradients for cocaine and ecgonines with cannabinoids result, as shown in FIG. 19, in absorption maxima at 259 nm, at 266 nm and 268.5 nm.

FIG. 20 shows the result of the formation of pairwise gradients for the cannabinoids. Maximum absorptions can be seen at 279 nm and at 284 nm. The maximum at 305 nm is the result of the additional occurrence of a benzodiazepine.

FIG. 21 shows that the formation of pairwise gradients of the non-linear 2nd derivatives for the group of amphetamines results in specific maxima at 278.5 nm and in the region of 307 nm. The existing concentrations permit the unambiguous quantification of the groups of substances.

FIG. 22 illustrates the reflection-spectroscopic properties of the racemic mixture consisting of equal proportions of the pure L-methadone enantiomer and the pure D-methadone enantiomer. L-methadone exhibits a single-peak maximum in the wavelength region of 256 nm. D-methadone and DL-methadone exhibit two-peak light spectra: D-methadone at 238 nm and at 256 nm, DL-methadone at 254.5 nm and at 350 nm. The D-enantiomer additionally exhibits a superposed characteristic spectrum in the wavelength region of 302 nm.

The generation of a reflected light spectrum according to the invention makes it possible reliably to detect psychoactive substances in extremely small substance concentrations. It is possible clearly to separate the individual spectra, which are superposed to form a composite reflected light spectrum. The evaluation results are available after an extremely short evaluation time. The reflected light spectrum can advantageously be recorded in non-invasive manner. This results in a low-cost and particularly reliable screening method for psychopharmacological agents (neuroleptics, antidepressants, sedatives and hypnotics), antiepileptics and antibiotics as well as metabolites thereof.

The selection method according to the invention provides the possibility to detect the presence of illegal substances and drugs of the aforementioned groups of drugs in the human organism without blood withdrawal, hair analysis or urine testing. It has become apparent from the investigations conducted in connection with the testing of the solution according to the invention that the method according to the invention is suitable for the routine detection of illegal substances in the blood. Consequently, the analysis method, which can be performed with a comparatively low-cost hardware set-up, is suitable for routine road traffic controls as well as for the detection of illegal drugs and other medicines and psychoactive substances which impair a person's fitness to drive.

Since the method according to the invention allows the reliable detection of both the substances themselves and also the concentrations thereof, it is also possible to make a qualitative distinction within groups of substances. In particular, it is also possible reliably to detect metabolites of the psychoactive substances. In this respect, the method according to the invention is especially suitable for the detection of cannabinoids, amphetamines and cocaine. Particularly in concentrations relevant with regard to a person's fitness to participate in road traffic, the psychoactive substances can routinely be detected by exposing the person's skin to light. A further area of application is the monitoring of the level of antiepileptics which are used in level-monitored manner in humans. There are drug-monitoring-based pharmacotherapeutic intervention possibilities in the field of antibiotics treatment and drug-monitoring-based psychopharmacotherapy with neuroleptics and antidepressants. The therefrom resulting user-specific dosage possibilities form, as such, the basis for a drug treatment which is matched to the needs of the individual patient. Such a treatment may also of itself be essential to the invention. The risks of over- and under-dosage are in this manner eliminated.

The particularly reliable evaluation of the in vivo recorded reflection spectra is accomplished preferably by a central evaluation unit. The data records indicative with regard to the recorded reflected light spectrum can be transmitted by a mobile communication means to a central computer, possibly over the Internet. The central computer is capable of executing extensive correlation algorithms in order to calculate from the reflected light spectrum the type and concentration of any illegal substances in the human organism, it being possible likewise to take account of known matrix effects.

On the basis of the detection and quantification of groups of substances made possible by the method according to the invention, it is possible to assess the momentary physiological condition of the person under examination. It has been shown that the method according to the invention also makes it possible reliably to detect metabolites of the psychoactive substances transdermally in the capillary bed. The method according to the invention is based on the principle that light of different wavelengths penetrates to different depths into the vital tissue. In particular, longer-wave (lower-energy) light penetrates deeper into the tissue than shorter-wave (higher-energy) light. This means that, at the same measuring point, short-wave light reaches less substance than long-wave light. Within the spectrum from short-wave to long-wave light one obtains a concentration gradient. This effect is used in advantageous manner by the measuring method according to the invention for determining the concentration of psychoactive substances, particularly chromophores, in the tissue.

If, now, the same measuring point is illuminated with light of different intensity, one obtains spectra originating from different depths. If these spectra are correspondingly weighted and subtracted from each other, one obtains the concentrations of certain substances as a function of the difference in depth.

This means that, on a defined wavelength which is characteristic of the substance only within a certain wavelength range, there is a defined change of intensity for each change of concentration. Assuming that the optical properties of the tissue surrounding the substance are constant, the associated light path length can be determined directly from the differences of intensity and distribution. Thus, a difference of distribution corresponds in advantageous manner to a certain difference of concentration. From the difference of light intensity associated with the difference of concentration it is possible to determine the path length according to Lambert-Beer's law.

The assumption of constant optical properties of the tissue in the measured volume was made above only for the purpose of simplification. By means of suitably differentiated algorithms it is also possible to take account of changes in the optical properties of the tissue surrounding the substance, as is frequently the case in practice.

The method according to the invention makes it possible—in the stratum corneum, in the epidermis and down into the dermis—qualitatively to determine substances even when inhomogeneously distributed in a measured volume of, say, 10 μm thickness. Consequently, this method is suitable for making quantitative statements on the concentration of a substance at a defined penetration depth. The depth resolution is preferably around 8 μm. This means that the skin can be examined in layer thicknesses of 8 μm from outside to inside, quantitatively and non-invasively by spectroscopic means.

According to the invention, the method of the invention is implemented by a spectral photometer, the spectral resolution of which is 0.33 nm. This makes it possible to perform 3 measurements per nm, with the result that each spectrum can be resolved and calculated at very small intervals. This makes it possible to differentiate substances with similar spectra. This high resolution can be advantageously achieved by a CCD array with 2048 cells.

The light which is radiated onto the person under examination is generated preferably using a deuterium lamp and a halogen lamp, which provide sufficient light in the range from 200 to 800 nm. It has been demonstrated that the quantities of drugs occurring in vivo amount in the tissue to around 0.05% of the total absorption. Therefore, the fluctuations in intensity of the light emitted from the lamp should be considerably smaller than the changes in absorption through the drugs. The voltage and current stability of the lamp is preferably less than 6×10⁻⁶ (p-p) and the current and voltage drift is preferably less than 0.01% per hour.

In order to change the light intensity, it is possible advantageously to employ an attenuating element capable of continuously changing the intensity of the incident light by means of a mechanically adjustable shutter. Using an SMA connector, the light source is connected to the attenuator by means of a fibre-optic cable. An optical system allows parallel light to be projected onto the output of the attenuator, which is likewise formed by an SMA connector. From there, the light is directed to the object via an optical fibre. Situated between the two SMA connectors is preferably a shutter which can be adjusted by means of a stepper motor. The shutter aperture is preferably calculated such that the characteristic of the light is not essentially influenced when the shutter is operated. The stepper motor is preferably controlled by a computer which also accommodates the optical recording unit. The motor is preferably of such design that it is capable of executing 10,000 steps per revolution. This allows the light intensity to be changed in sufficiently fine steps in order to achieve adequate local resolution at different depths.

Alternatively to the above-described measure, it is also possible to achieve defined changes of the light intensity by using LED light sources, without changing the radiation characteristics of the emitted light. In this case, it is possible to dispense with the above-described control of the shutter achieved by, for example, a stepper motor.

A sensor head has been developed for implementing the method according to the invention through reflected light measurement on the human skin. Said sensor head has preferably 19 optical fibres which apply the light to the skin. Four optical fibres collect the reflected light and direct it to a CCD array. The quality of the measured values can be further improved by using an optical fibre having an even greater number of optical fibres, such as 70, this permitting an even more homogeneous illumination of the tissue.

Illegal drugs, psychopharmacological agents (neuroleptics, antidepressants, sedatives and hypnotics), antiepileptics and antibiotics can be detected by the method according to the invention, since it has been demonstrated that, particularly in a wavelength range from 200 to 800 nm, a multiplicity of the said substances cause a clear change of the reflected light spectrum obtained in vivo. On the basis preferably of pure spectra generated with regard to the substances to be detected as well as in consideration of the solution behaviour thereof, through consideration of the influence of the solvents on any displacements of the reflected light spectra, through consideration of photo-effects, spectroscopically relevant interactions between the respective substances, their absorption properties in different physiological media and their transcutaneous measuring characteristics it is possible yet further to enhance the reliability of the analysis result.

### Detection of Methadone/polamidone

Methadone occurs in two stereo-enantiomer forms. The one molecular form—referred to in the following as D-methadone—turns polarized light to the right, while the other—referred to in the following as L-polamidone—turns it to the left. The form which is effective analgesically and for compensation of withdrawal symptoms is predominantly the L-form. Since, in medical/therapeutic practice, one encounters both a mixture of both forms and also L-polamidone in its pure form, it is important to be able to distinguish the two methadones.

FIG. 22 shows that the spectra of D-methadone, L-methadone and the mixture of both are in some cases very similar while they are clearly different in other spectral ranges. The difference between them permits the unambiguous discrimination of the enantiomers.

The method according to the invention can be implemented as follows:

In order to examine a person, a sensor head is applied to the surface of their skin, for example in the region of the inner arm. The sensor head radiates light in a wavelength range from 240 to 800 nm into the skin. The light reflected from a certain depth of the skin is captured by an optical fibre means and is supplied to a spectrometer. The spectrum of the reflected light captured by the optical fibre is recorded. The recorded spectrum is subjected to a mathematical evaluation procedure by a computer means. This mathematical evaluation procedure takes account preferably of certain correlation properties between spectra which are indicative of a multiplicity of potentially relevant drugs and medicines. Since the reflected light spectrum captured by the spectrometer is unambiguously indicative not only of the type of the substance interacting with the light, but also of the concentration thereof, it is possible, on the basis of the reference spectra available for a multiplicity of substances, to calculate from the reflected light spectrum the type and concentration of the substances to be detected.

Both the light source for generating the examination light and also the sensor head for capturing the reflected light, including the spectrometer, are preferably in the form of mobile handheld devices. It is possible, for evaluation of the recorded reflected light spectrum, to transmit said spectrum to a central evaluation unit, for example via a mobile communication system. The thus transmitted reflected light spectrum can then be evaluated on the basis of an extensive data record as well as by powerful hardware. The thus obtained evaluation result can then be transmitted back, for example in the form of an SMS data record, into the area of the capturing means.

It is also possible for detection and evaluation to be performed online at the examination device itself, for example in road traffic, in doctors' surgeries or in in-patient treatment units in hospitals. In this case, the person carrying out the examination receives the measurement results directly printed out on a connected printer.

Furthermore, oxygen can be determined intraoperatively online without the need for invasive oxygen measurements by blood gas analysis.

In paediatrics, oxygen, haemoglobin and drug level can be determined without the need for the traumatizing and technically complex withdrawal of blood.

In emergency medicine, acute intoxication by drugs and medicines can be diagnosed in an extremely short time and can be specifically treated more quickly than hitherto.

FIG. 23 shows the fluorescence spectrum of cocaine. At a wavelength of the excitation light of 270 nm there is a steep rise between 230 and 330 nm. Thereafter, the spectrum falls off continuously and reaches a 0-value at 730 nm. At a concentration of 0.01 mg/ml there is an optical density of around 2080.

FIG. 24 shows the fluorescence spectrum of LSD. At a wavelength of the excitation light of 310 nm there is a steep rise between 340 and 350 nm. Thereafter, the spectrum falls off continuously and regains the starting value at 450 nm. At a concentration of 0.01 mg/ml there is an optical density of around 10,000.

FIG. 25 shows the fluorescence spectrum of 11 hydroxy-THC. At a wavelength of the excitation light of 300 nm there is initially a steep pointed peak between 360 and 400 nm with a maximum at 390 nm. Starting from 430 nm there is a flatter broader fluorescence spectrum which regains its starting value at around 800 nm. At a concentration of 0.01 mg/ml there is an optical density in the first spectrum of around 4100. The second spectrum reaches the maximum value at an optical density of around 490.

FIG. 26 shows the fluorescence spectrum of heroin. At a wavelength of the excitation light of 255 nm there is a first maximum at around 380 nm and then a reduction of the signal at around 430 nm and a second maximum at around 470-480 nm. In the further course of the signal the fluorescence spectrum falls and reaches its minimum after an inhomogeneous fall-off at around 420 nm.

What is claimed is:

1. Method for the detection of substances in vital tissue in which
light of a predetermined wavelength is directed onto the tissue in such a manner that the light penetrates into the tissue;
at least some of the light escaping from the tissue is captured and the reflected light is analyzed with an association being made between its wavelength and intensity; and
the thus determined properties of the reflected light are compared with at least one reference system, the presence and/or concentration of a substance being deduced on the basis of a correlation with the reference system.

2. Method according to claim 1, characterized in that the reference system makes available reference values, each of which is significant for a certain substance or group of substances, the intensity distribution of the light over its wavelength being compared with said reference values.

3. Method according to claim, characterized in that a multiplicity of reference spectra is provided, and in that the presence and/or concentration of a substance or group of substances is deduced depending on the fulfillment of predetermined relationships between the two or more reference spectra and the measured intensity distribution.

4. Method according to claim 2, characterized in that the concentration of a detected substance is deduced on the basis of the intensity of selected wavelength regions.

5. Method according to claim 1, characterized in that the excitation spectrum is in the range from 200 to 800 nm.

6. Method according to claim 1, characterized in that the frequency of the excitation light used for excitation is outside of the measured reflectance spectrum.

7. Method according to claim 1, characterized in that the comparison of the measured wavelengths/intensity distribution is performed on the basis of a correlation consideration.

8. Device for the detection of substances in vital tissue, with a light source for producing light of a defined wavelength and for radiating the light onto the vital tissue in such a manner that the light penetrates into the tissue; with a light-capturing means for capturing at least some of the light reflected from the tissue; with a light-intensity-measuring means for measuring the intensity of the reflected light with an association being made with the wavelength; and with a correlation-determining means for determining correlation features of the calculated intensity distribution of the reflectance spectrum with at least one reference system, the presence and/or concentration of a substance being deduced on the basis of the determined correlation features.

9. Device according to claim 8, characterized in that an optical fibre means is provided, for directing the light emitted by the light source onto the tissue.

10. Device according to claim 9, characterized in that an optical fibre means is provided, for capturing the reflected light.

11. Device according to claim 10, characterized in that a spectral dispersion means is provided, for splitting the reflected light into its spectral components.

12. Device according to claim 11, characterized in that a CCD recording means is provided, for determining the intensity of the individual spectral components.

13. Device according to claim 8, characterized in that a means is provided, for capturing the light from a predetermined measuring depth.

14. Device according to claim 13, characterized in that a plurality of reflectance spectra are captured for different, defined measuring depths.

15. Device according to claim 8, characterized in that a storage means is provided, and in that a reference system in the form of a reference data record is stored in said storage means.

16. Device according to claim 15, characterized in that the reference data record contains characteristic data with regard to the substance-specific spectra.

17. Device according to claim 8, characterized in that the correlation-determining means makes available a plurality of correlation criteria for the analysis of the spectra.
